(19) United States
(12) Patent Application Publication
(10) Pub. No.: US 2006/0263876 A1
(43) Pub. Date: Nov. 23, 2006

(54) Neural crest stem cells and uses thereof

(76) Inventors: Freda Miller, Toronto (CA); Ian McKenzie, Toronto (CA)

Correspondence Address: CLARK & ELBING LLP, 101 FEDERAL STREET, BOSTON, MA, 02110, US

(21) Appl. No.: 11/295,370

(22) Filed: Dec. 6, 2005

Related U.S. Application Data
(63) Continuation-in-part of application No. PCT/CA04/00820, filed on Jun. 7, 2004.

(51) Int. Cl. C12N 5/08

(52) U.S. Cl. 435/325; 435/368

(57) ABSTRACT

This present invention features methods and composition for the isolation and proliferation of neural crest stem cells (NCSCs) from embryonic tissues as well as from tissues from a post-natal mammal. According to this invention, NCSCs are capable of producing non-neuronal and neuronal cells under the appropriate conditions. The cells of the invention therefore provide an accessible source for autologous and heterologous transplantation into the central nervous system, the peripheral nervous system, as well as other damaged tissues.

### CROSS-REFERENCE TO RELATED APPLICATIONS

This application is a continuation-in-part of International Application No. PCT/CA2004/000820, filed Jun. 7, 2004, which claims the benefit of U.S. Provisional Application No. 60/476,772, filed Jun. 6, 2003, both of which are incorporated herein by reference in their entirety.

### BACKGROUND OF THE INVENTION

Various disease states, such as cardiovascular, neurological, and muscular diseases, are characterized by the irreversible loss of cells. Because cells that are destroyed in such conditions are often non-renewable, these diseases are often debilitating and incurable. Parkinson's disease, for example, is a progressive neurodegenerative disorder of unknown cause. In healthy brain tissue, dopaminergic neurons extend from the substantia nigra of the brain into the neighboring striatum. In Parkinson's disease however, these dopaminergic neurons die and cannot be replaced.

Stem cells are undifferentiated cells that exist in many tissues of embryos and adult mammals. In embryos, blastocyst stem cells differentiate to form the specialized tissues and organs of the developing fetus. In adults, specialized stem cells in individual tissues provide a source of cells for the replacement of cells lost as a result of natural attrition, disease, or injury. Due to their multipotency and self-renewing nature, stem cells may therefore be used as starting material for the production of cell types to replenish lost tissue material in cases in which a disease, disorder, or abnormal physical state has destroyed or damaged normal tissue. However, the progress of stem cell transplantation has been impeded by difficulties in isolating sufficient numbers of stem cells and maintaining these cells in culture for a sufficient amount of time while still retaining their multipotent state.

Thus, there is a clear need to develop methods for isolating and proliferating stem cells in vitro in order to replace damaged or diseased tissue.

### SUMMARY OF THE INVENTION

In general, the present invention provides methods and compositions for the isolation and proliferation of neural crest stem cells (NCSCs).

The present invention is based on our discovery that NCSCs can be substantially purified from embryonic tissues as well as tissues from postnatal mammals. Most importantly, we provide methods for the purification of NCSCs that can subsequently be maintained in culture for extended periods of time, a significant advantage relative to previous methods. These NCSCs possess desirable features in that they are multipotent and self-renewing. Under appropriate conditions, these NCSCs differentiate into neuronal cells (e.g., neurons and glial cells such as oligodendrocytes, Schwann cells, and astrocytes), non-neuronal cells (e.g., cardiomyocytes, lung cells, adipocytes, pancreatic islet cells, hematopoeitic cells, kidney cells, hepatocytes, chondrocytes, epithelial cells, endothelial cells, skeletal muscle cells, melanocytes, or smooth muscle cells), cartilage, or connective tissue. Thus, the present invention is particularly useful to treat, prevent, or reduce diseases that are characterized by the loss of a cell type. In this regard, the NCSCs of the invention may be used for autologous or heterologous transplants to treat, for example, diabetes as well as neurodegenerative, cardiovascular, or muscular diseases, disorders, or abnormal physical states.

Accordingly, in the first aspect, the invention features a mammalian NCSC capable of producing non-neuronal and neuronal cells and expressing p75, PSA-NCAM, and nestin. The invention also features a mammalian NCSC capable of producing non-neuronal and neuronal cells that expresses PSA-NCAM and nestin but that does not express p75. The NCSC of the invention may also express one, two, three, four, five, or all of the following molecular markers: FGFR, CD44, S100β, Pax3, twist, and fibronectin.

In another aspect, the invention features pharmaceutical compositions that include a cell population containing NCSCs. The cell population of the invention typically contains at least 10 cells, of which at least 30%, 40%, 50%, 60%, 70%, 80%, 90%, 95%, or even 100% of cells are mammalian NCSCs capable of producing non-neuronal and neuronal cells. In this population, at least 50%, 60%, 70%, 80%, 90%, 95%, or even 100% of NCSCs express p75, PSA-NCAM, and nestin. Alternatively, at least 20%, 30%, 40%, 50%, or more than 50% of NCSCs express PSA-NCAM and nestin but do not express p75.

The NCSC may be obtained, for example, by a method that includes the steps of: (a) culturing a mammalian tissue containing NCSCs in a first culture for a period of at least two days under conditions in which NCSCs adhere to the culture surface (e.g., poly-D-lysine and fibronectin); (b) transferring adherent cells from step (a) to a second culture under conditions in which NCSCs grow non-adherently and non-NCSCs grow adherently or die; and (c) collecting nonadherent cells. Typically, cells are cultured in the presence of growth factors such as FGF, EGF, and B27. According to this invention, the nonadherent cells obtained from step (c) are NCSCs that may be cultured for a period of 5, 10, 20, 30, 40, 50, 70, 80, or 100 days. Using this method, at least 20, 50, 100, 1000, 10,000, 50,000, 100,000, 500,000, or 1,000,000 NCSCs may be obtained. In addition to humans, the NCSC may be isolated from any mammal (e.g., mouse, rat, cat, dog, horse, baboon, or pig) and may be isolated from embryonic tissue (e.g., neural tube) or from tissues of a post-natal mammal (e.g., tissues of the gastrointestinal tract). Typically, the NCSC is obtained by a method that does not employ an antibody specific to p75. If desired, the adherent cells obtained from step (c) may further be cultured under conditions in which NCSCs produce neurons, glial cells (e.g., oligodendrocytes, Schwann cells, and astrocytes), cardiomyocytes, lung cells, adipocyte, pancreatic islet cells, hematopoeitic cells, kidney cells, hepatocytes, chondrocytes, epithelial cells, endothelial cells, skeletal muscle cells, melanocytes, smooth muscle cells, cartilage, or connective tissue. In the presence of serum for example, NCSCs differentiate into smooth muscle cells. In the presence of BMP2, neurons are produced whereas the addition of HRG-β induces glial differentiation. Accordingly, the present invention is particularly useful for the treatment of diseases that characterized by the failure of a cell type by administering to a mammal in need thereof the NCSCs of the invention, or alternatively, cells that have differentiated from such NCSCs. If desired, the NCSC of the invention may also express a heterologous gene, encoding therapeutic proteins for example. The heterologous gene may be in an expression vector. The heterologous gene may also be operably linked to an inducible promoter.

By “a disease characterized by failure of a cell type” is meant one in which the disease phenotype is the result of loss of cells of that cell type or the loss of function of cells of that cell type.

By “expression vector” is meant a DNA construct that contains a promoter operably linked to a downstream gene, cistron, or RNA coding region (e.g., an antisense RNA coding region). Transfection of the expression vector into a recipient cell allows the cell to express RNA encoded by the expression vector. An expression vector may be a genetically engineered plasmid or virus, derived from, for example, a bacteriophage, adenovirus, retrovirus, poxvirus, herpesvirus, or artificial chromosome.

By “neural crest stem cell” (NCSC) is meant a cell derived from the neural crest having the ability of self-renewal and of asymmetrical division. A NCSC of the invention is capable of dividing to produce two different daughter cells, one of which is has the potential of the parental cell, and the other being a cell having a more restricted developmental potential relative to the parental NCSC. For example, the more restricted cell has or may have characteristics of a neuron, glial cell (e.g., oligodendrocyte, Schwann cell, and astrocyte), cardiomyocyte, lung cell, adipocyte, pancreatic islet cell, hematopoeitic cell, kidney cell, hepatocyte, chondrocyte, epithelial cell, endothelial cell, skeletal muscle cell, melanocyte, or smooth muscle cell. The NCSC of the invention may also produce cartilage or connective tissue. Not every cell division need be an asymmetrical division. It is possible that a given division may result in two multipotent cells or two developmentally restricted progeny only. NCSCs can be isolated from embryonic tissue (e.g., neural tube) or from adult tissues (e.g., gastro-intestinal tract).

By “operably linked” is meant that a nucleic acid molecule and one or more regulatory sequences (e.g., a promoter) are connected in such a way as to permit expression and/or secretion of the product (i.e., a polypeptide) of the nucleic acid molecule when the appropriate molecules (e.g., transcriptional activator proteins) are bound to the regulatory sequences.

By a “population of cells” is meant a collection of at least ten cells. Preferably, the population consists of at least twenty cells, more preferably at least one hundred cells, and most preferably at least one thousand, or even one million cells. Because the NCSCs of the present invention exhibit a capacity for self-renewal, they can be expanded in culture to produce populations of even billions of cells.

By “postnatal” is meant an animal that has been born at full term.

By “therapeutic protein” is meant a protein that improves or maintains the health of the cell expressing the protein or of a cell in proximity to the cell expressing the therapeutic protein. Example therapeutic proteins include, without limitation, growth factors (NGF, BDNF, NT-3, NT-4/5, HGF, TGF-β family members, PDGF, GDNF, FGF, EGF family members, IGF, insulin, BMPs, Wnts, hedgehogs, and heregulins), cytokines (LIF, CNTF, TNF, interleukins, and gamma-interferon), and anti-apoptotic proteins (IAP proteins, Bcl-2 proteins, Bcl-X_{L}, Trk receptors, Akt, PI3 kinase, Gab, Mek, E1B55K, Raf, Ras, PKC, PLCγ, FRS2, rAPs/SH2B, and ΔNp73).

### DESCRIPTION OF THE FIGURES

FIG. 1 is a photograph of the neural tube explant.

FIG. 2 is a series of photographs showing neural crest stem cells (NCSCs) growing adherently and non-adherently.

FIG. 3 shows a series of immunostains of central nervous system (CNS)-derived neurospheres, neural crest-derived spheres (NC), and skin-derived precursors (SKPs) using an antibody specific to PSA-NCAM, CD44, and FGF-R.

FIG. 4 shows a series of immunostains of CNS-derived neurospheres, NC-derived spheres, and SKPs using an antibody specific to S100β, fibronectin, and nestin.

FIG. 5 is a picture showing an RT-PCR analysis to detect the expression of Pax-3 and Twist in the neural tube, CNS-derived neurospheres, NC-derived spheres, and SKPs.

FIG. 6 shows an immunostain of an NC-derived sphere using an antibody specific to p75.

FIG. 7 shows an immunostain of NC-derived spheres (plated on poly-D-lysine and laminin and cultured in the presence of serum) using an antibody specific to neurofilament.

FIG. 8 shows a series of immunostains of NC-derived spheres (plated on poly-D-lysine and laminin and cultured in presence of N2; N2 and BMP2; and serum and BMP2) using antibodies specific to neurofilament and smooth muscle actin.

FIGS. 9A and 9B show a series of immunostains of NC-derived spheres following treatment with BMP2 (9A) or HRG-β (9B) using antibodies to neurofilament or CNPase and GalC.

FIG. 9C is a graph showing the percentage of neurons and glial cells produced as a result of treating neural crest stem cells with BMP2 or HRG-β.

### DETAILED DESCRIPTION

In general, the present invention provides methods and compositions for the isolation and proliferation of mammalian neural crest stem cells (NCSCs) as well as their differentiation into non-neuronal and neuronal cells.

This invention is based on our discovery that NCSCs can be substantially purified from mammals (e.g., humans). According to the present methods, the neural tube, for example, is initially cultured for a period of at least a half hour, one hour, two hours, four hours, six hours, 24 hours, or more in a first culture vessel under conditions in which NCSCs can migrate out of the neural tube and attach to the culture substrate. In this particular step, the media may be supplemented with growth factors such as N2, chick extract, retinoic acid, BDNF, and FGF while the culture substrate may be coated with poly-D-lysine and fibronectin, for example. Cells that have adhered to the culture substrate are next collected by trypsinization and transferred to a second culture vessel under conditions in which NCSCs can attach to the culture substrate (e.g., poly-D-lysine and fibronectin). In this step, cells are cultured in media supplemented with FGF2, EGF, and B27, for example. After a period of two days, three days, four days, one week, or two weeks, adherent cells are collected and transferred to a third culture vessel under conditions in which NCSCs grow non-adherently (e.g., non-coated plastic). NCSCs will typically assemble into sphere-like conformations and grow three-dimensionally. Non-adherent cells are therefore collected and tested for the expression of NCSC-specific markers while cells that attach to the culture vessel or that die are discarded. According to this invention, the substantially purified NCSCs of the invention express the NCSC-specific markers PSA-NCAM and nestin and may or may not express p75. In addition, these cells may also express one or more NCSC-specific markers, such as FGF-R, CD44, S100β, Pax3, twist, and fibronectin. Using the methods of the invention, the substantially purified NCSCs may be cultured for extended periods of time while still retaining their self-renewing and mutipotent properties, a significant advantage over previous methods. Thus, NCSCs may be cultured for at least 5, 10, 20, 30, 40, 50, 70, 80, 100 days, or longer (e.g., one year or more). Due to their multipotency, the NCSCs of the invention proliferate in culture such that large numbers of stem cells can be generated.

According to this invention, in addition to embryonic tissues such as the neural tube, NCSCs may also be isolated from tissues of post-natal mammals, including, for example, gastro-intestinal tissues. Desirably, at least 30%, 40%, 50%, 60%, 70%, 80%, more preferably 90%, even more preferably 95%, and even 100% of the cells that are purified are NCSCs. Preferably, the population of cells isolated is at least 20 cells, 50 cells, 100 cells, 1000 cells, 10,000 cells, 50,000 cells, 100,000 cells, 500,000 cells, 1,000,000 cells, or more.

Under the appropriate conditions, the NCSCs of the invention may also differentiate into various cell types. Thus, NCSCs may produce non-neuronal cells (e.g., cardiomyocytes, lung cells, adipocytes, pancreatic islet cells, hematopoeitic cells, kidney cells, hepatocytes, chondrocytes, epithelial cells, endothelial cells, skeletal muscle cells, melanocytes, or smooth muscle cells) and neuronal cells (e.g., neurons, Schwann cells, oligodendrocytes, or astrocytes). Preferred neurons include neurons expressing one or more of the following neurotransmitters: dopamine, GABA, glycine, acetylcholine, glutamate, and serotonin. NCSCs may also produce cartilage or connective tissue. Thus, the present invention is particularly useful to treat, prevent, or reduce a disease characterized by the loss of a cell type by administering to a mammal in need thereof the NCSCs of the invention, or alternatively, the NCSC-differentiated cells. In this regard, the NCSCs of the invention are useful for generating cells for use, for example, in autologous transplants for the treatment of degenerative disorders or trauma (e.g., spinal cord injury). In one example, NCSCs may be differentiated into dopaminergic neurons and implanted in the substantia nigra or striatum of a Parkinson's disease patient. In a second example, the cells may be used to generate oligodendrocytes for use in autologous transplants for the treatment of multiple sclerosis. In another example, the NCSCs may be used to generate Schwann cells for treatment of spinal cord injury, cardiac cells for the treatment of heart disease, or pancreatic islet cells for the treatment of diabetes. In still another example, NCSCs may be used to replace cells damaged or lost to bacterial or viral infection, or those lost to traumatic injuries such as burns, fractures, and lacerations. If desired, in any of the foregoing examples, the cells may be genetically modified to express, for example, a growth factor, an anti-apoptotic protein, or another therapeutic protein. NCSCs may therefore by stably or transiently transfected with a heterologous gene, such as a gene encoding a therapeutic protein.

Pharmaceutical Compositions for Cell Therapy

The substantially purified NCSCs of the present invention may be used to prepare pharmaceutical compositions that can be administered to humans or animals for cell therapy. The cells may be undifferentiated (NCSC) or differentiated (e.g., cardiomyocytes, lung cells, adipocytes, pancreatic islet cells, hematopoeitic cells, kidney cells, hepatocytes, chondrocytes, epithelial cells, endothelial cells, skeletal muscle cells, melanocytes, smooth muscle cells, neurons, Schwann cells, oligodendrocytes, astrocytes, cartilage, or connective tissue) prior to administration. For example, NCSCs cultured in the presence of BMP2 and HRG-β differentiate into neurons and glial cells, respectively. Furthermore, serum induces smooth muscle cell differentiation. Methods to induce cellular differentiation are described in detail, for example, by Dupin et al. (An. Acad. Bras. Cien. (2001) 73(4): 535-45), Kruger et al. (Neuron (2002), 35:657-669), Kennea et al. (J. Pathol. (2002) 197(4): 536-550), and Takano et al. (Pigment Cell Research (2002) 15(3): 192-200). Such methods are further described in U.S Ser. Nos. 09/925,911, 09/946,325, 09/991,480, 10/112,939, 10/153,972, and 10/199,918, and U.S. Pat. Nos. 5,654,183, 5,672,499, 5,693,482, 5,733,727, 5,849,553, 5,942,225, 5,633,426, 6,497,872, and 6,528,245. All of these references are hereby incorporated by reference. PCT publication WO 99/16863, for example, describes the differentiation of forebrain multipotent neural stem cells into cells of the hematopoietic cell lineage in vivo. Because the NCSCs of the present invention are also multipotent, they are also capable of differentiating into non-neural cells types, such as hematopoietic cells.

Accordingly, a patient having a disease or disorder characterized by cell loss may be administered with the NCSCs of the present invention or with cells that have derived from NCSCs. Following such administration, NCSCs differentiate and eventually replace the cells lost in the disease or disorder. Furthermore, transplantation of NCSCs and their progeny provide an alternative to bone marrow and hematopoietic stem cell transplantation to treat blood-related disorders. Other uses of stem cells that are applicable to the present NCSCs are described in Ourednik et al. (Clin. Genet. (1999) 56:267-278), hereby incorporated by reference. Dosages to be administered depend on patient needs, on the desired effect, and on the chosen route of administration.

The invention also features the use of the cells of this invention to introduce therapeutic proteins into the diseased, damaged, or physically abnormal central nervous system, peripheral nervous system, or other tissue, as described, for example in U.S. Ser. Nos. 09/916,639 and 10/199,918, both of which are hereby incorporated by reference. In general, therapeutic proteins are proteins that improve or maintain the health of the cell expressing the protein or that of a cell in proximity to the cell expressing the therapeutic protein. Exemplary therapeutic proteins include, without limitation, growth factors (NGF, BDNF, NT-3, NT-4/5, HGF, TGF-β family members, PDGF, GDNF, FGF, EGF family members, IGF, insulin, BMPs, Wnts, hedgehogs, and heregulins) cytokines (LIF, CNTF, TNF, interleukins, and gamma-interferon), and anti-apoptotic proteins (IAP proteins, Bcl-2 proteins, Bcl-X_{L}, Trk receptors, Akt, PI3 kinase, Gab, Mek, E1B55K, Raf, Ras, PKC, PLCγ, FRS2, rAPs/SH2B, and ΔNp73). The NCSC or the NCSC-differentiated cell therefore acts as a vector to transport the therapeutic protein. In order to allow for expression of this protein, suitable regulatory elements may be derived from a variety of sources, and may be readily selected by one with ordinary skill in the art. Examples of regulatory elements include a transcriptional promoter and enhancer or RNA polymerase binding sequence, and a ribosomal binding sequence, including a translation initiation signal. Additionally, depending on the vector employed, other genetic elements, such as selectable markers, may be incorporated into the recombinant molecule. The recombinant molecule may be introduced into the NCSCs or the NCSC-differentiated cells using in vitro delivery vehicles such as retroviral vectors, adenoviral vectors, DNA virus vectors and liposomes. They may also be introduced into such cells in vivo using physical techniques such as microinjection and electroporation or chemical methods such as incorporation of DNA into liposomes. The genetically altered cells may be encapsulated in microspheres and implanted into or in proximity to the diseased or damaged tissue.

### EXAMPLE 1

### Isolation of Neural Crest Stem Cells from Neural Tube Explants

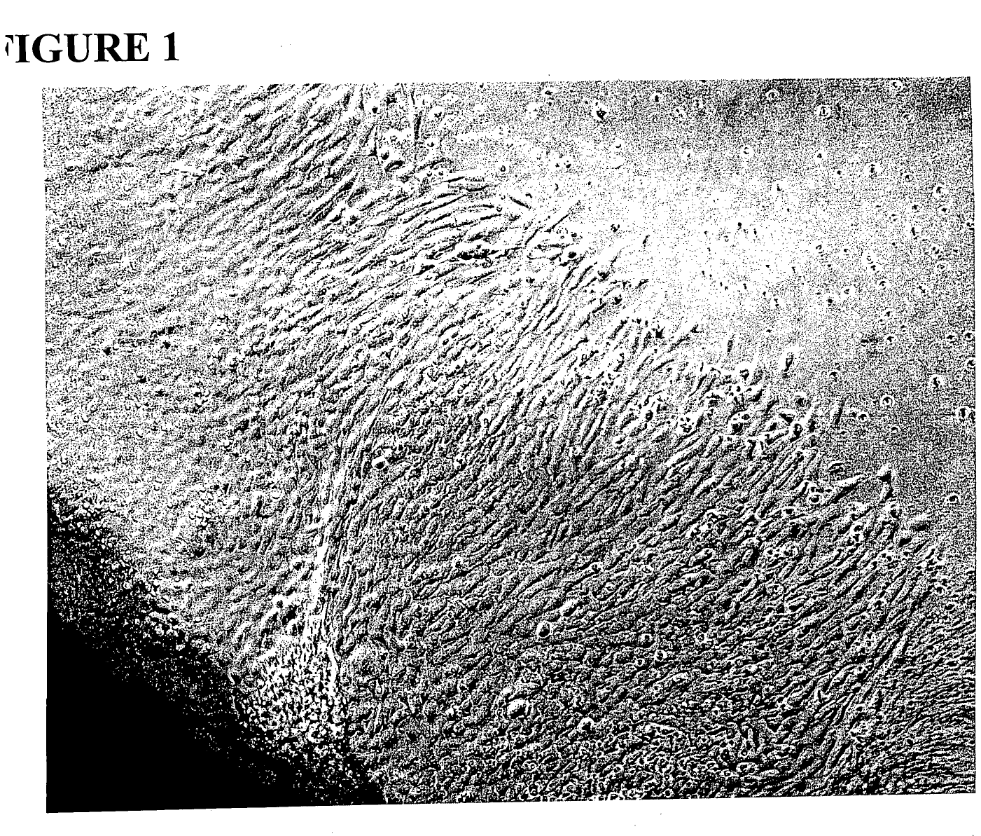

Neural tubes were obtained from E10.5 rat embryos (see FIG. 1). Following dissection, these tubes were placed on moist culture dishes that had previously been sequentially coated with poly-D-lysine and fibronectin. Dishes were placed in the incubator at 37° C. for 30 minutes to allow the tubes to adhere to the dishes. Following this incubation, dishes were flooded with 3:1 DMEM/F12 supplemented with N2, chick extract, retinoic acid, BDNF, and FGF. Tubes were cultured under these conditions for a period of four days. During this period, NCSCs migrated out of the tube and had attached to the substrate. The tubes and a margin of the attached migrating cells were scraped off the dish, which was then washed several times to remove all non-neural crest cells.

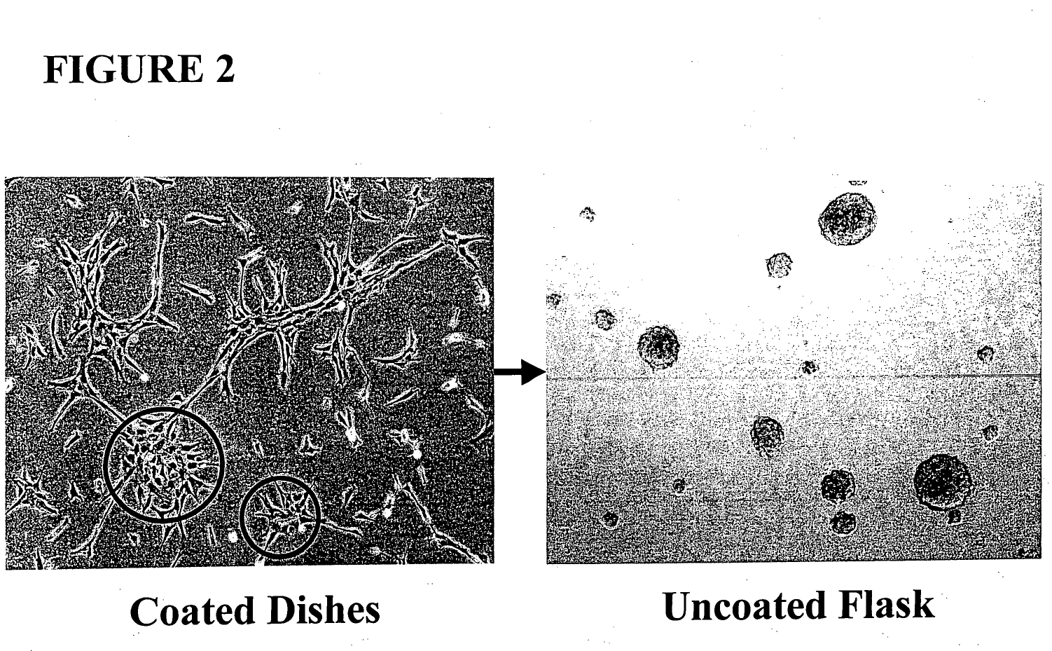

Cells that had adhered to the culture substrate were trypsinized, transferred to new culture dishes coated with poly-D-lysine and fibronectin, and cultured in the presence of DMEM:F12 supplemented with FGF2, EGF, and B27. Adherent cells, including NCSCs, proliferated under these conditions and formed colonies (see FIG. 2). When the dish was semi-confluent, cells were trypsinized and split into two new dishes using the previous conditions. The dish was again grown to semi-confluency. At this time, cells were trypsinized and placed in a non-coated culture dish containing the same media described above (nonadherent conditions).

After a period of one week, floating clusters of proliferating cells could be observed (see FIG. 2). These floating spheres were then mechanically dissociated and split into three new flasks. Under these non-adherent conditions and in this media, NCSCs grow as three-dimensional structures. Cells from floating spheres were therefore analyzed for the expression of NCSC-specific markers. Cells have been passaged 10 times in this manner.

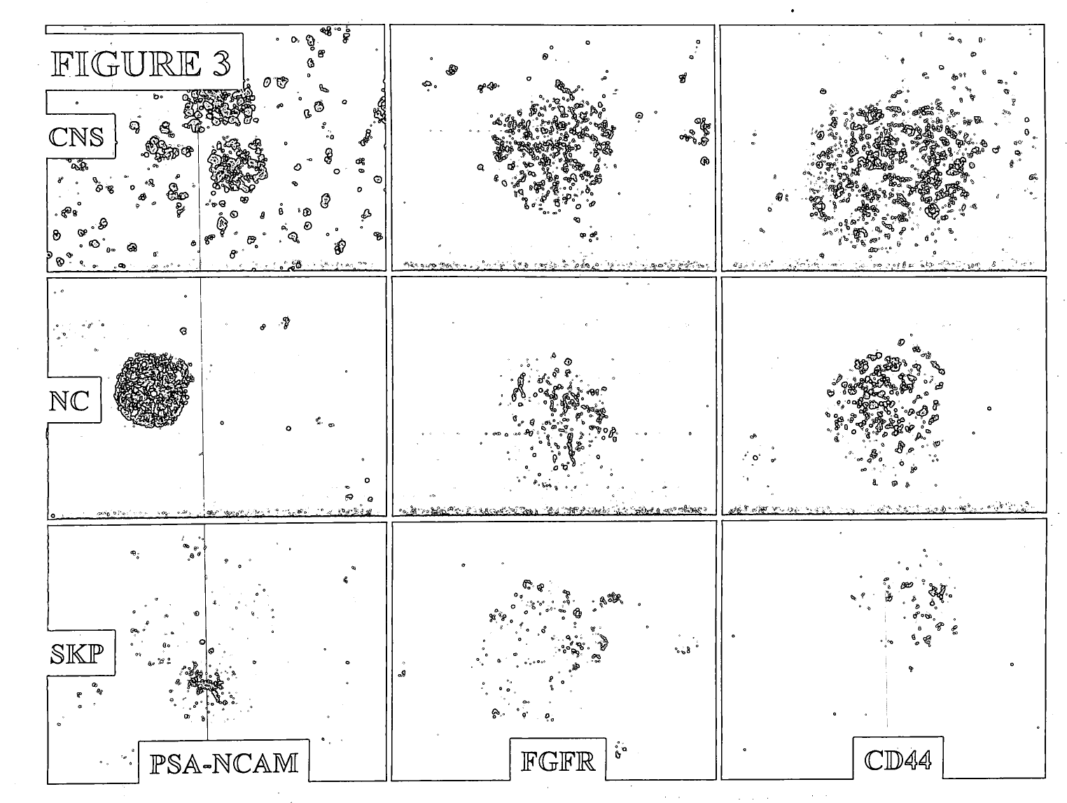

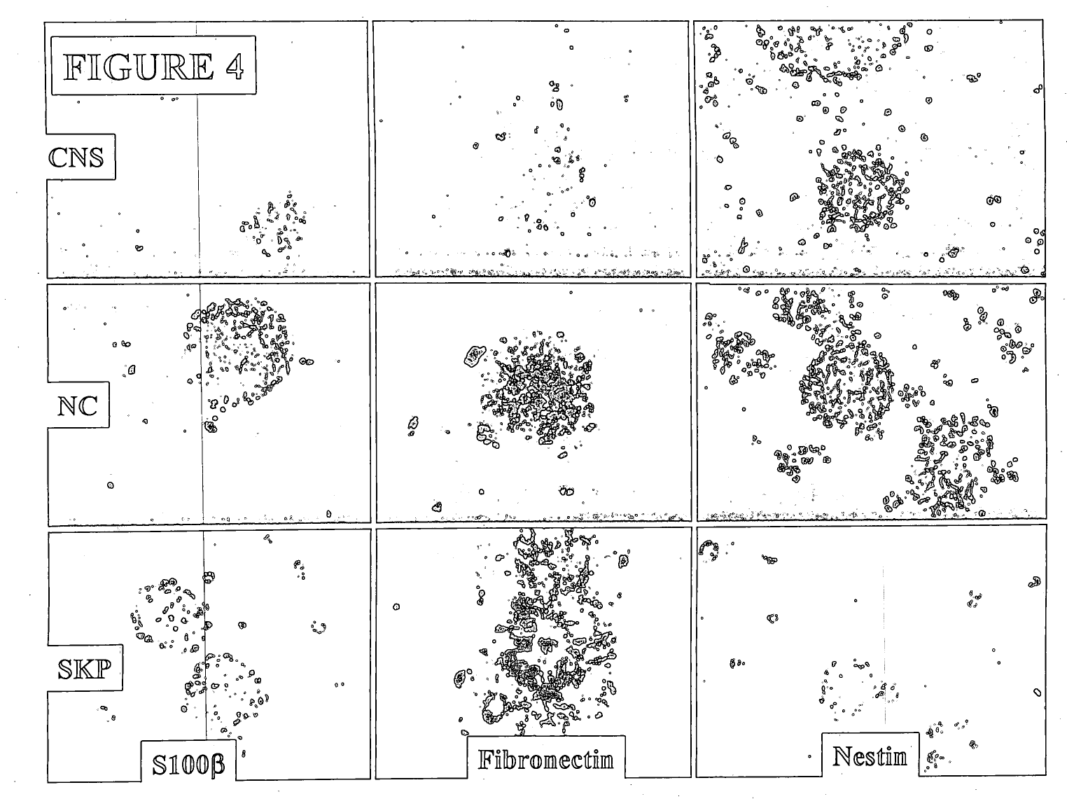

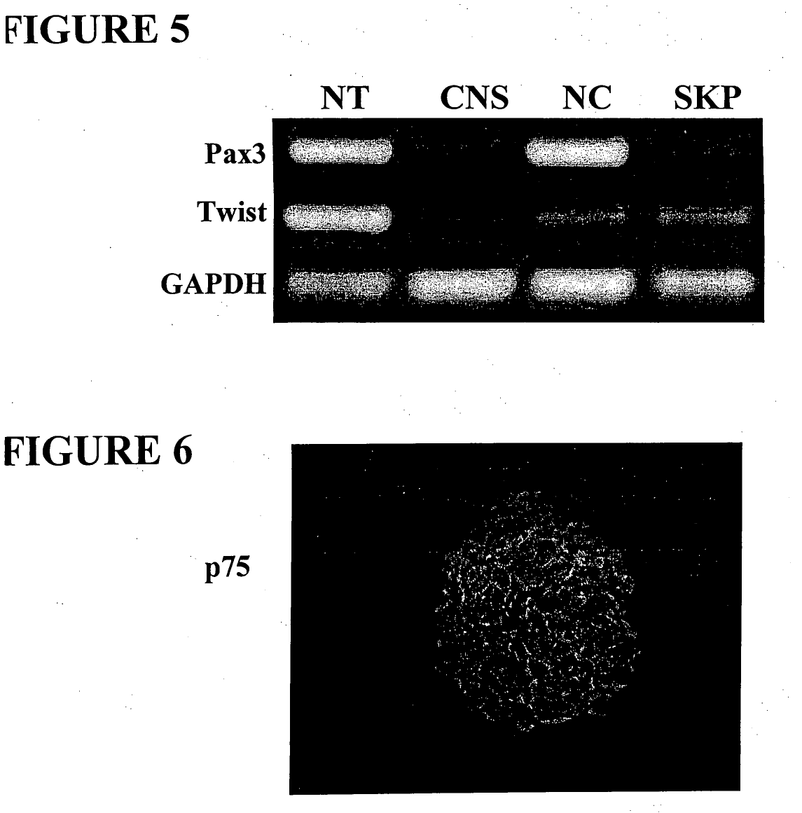

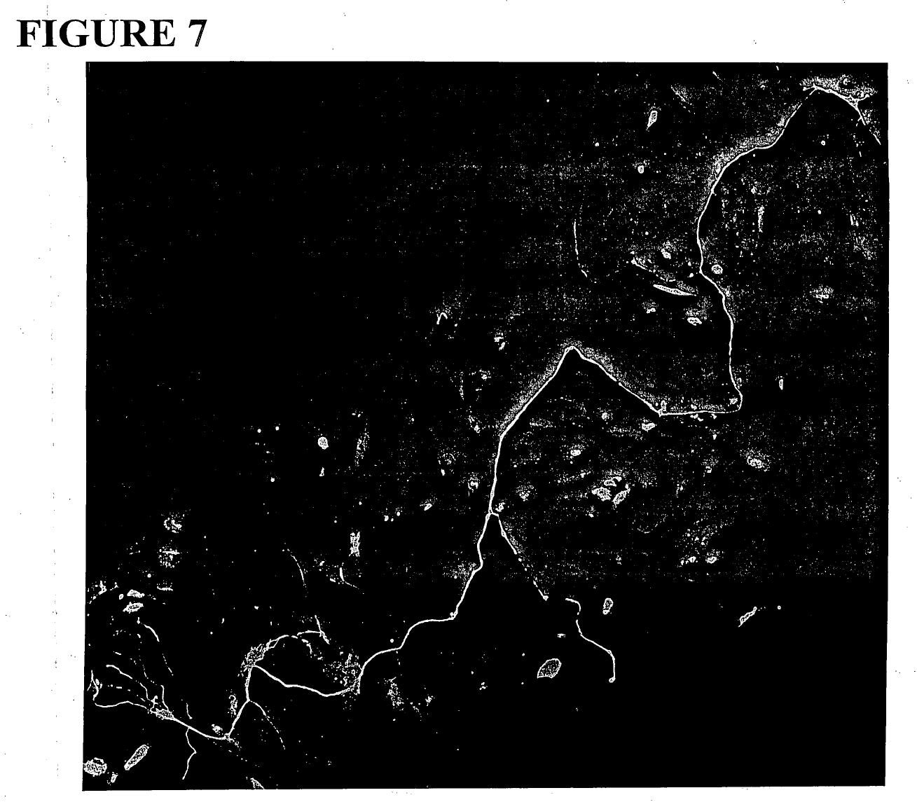

CNS-derived neurospheres, neural crest-derived spheres, and skin-derived stem cells (SKPs) were spun down on cytospin slides for immunocytochemical analysis. Antibodies were used to detect PSA-NCAM, FGFR, CD44, S100β, fibronectin, and nestin. NC-derived spheres were spun down on cytospin slides and immunostained for p75. As shown in FIGS. 3 and 4, NCSCs expressed PSA-NCAM, nestin, and fibronectin. In contrast, SKPs did not express PSA-NCAM. Cells of the neural crest-derived spheres were both p75 positive and p75 negative (FIG. 6). cDNA was also generated from each cell type, and the mRNA levels of Pax-3 and Twist were determined (FIG. 5). Compared to other cells, cells of the NC-derived spheres expressed high levels of Pax-3 and Twist.

### EXAMPLE 2

### Differentiation from NCSCs-Derived Spheres

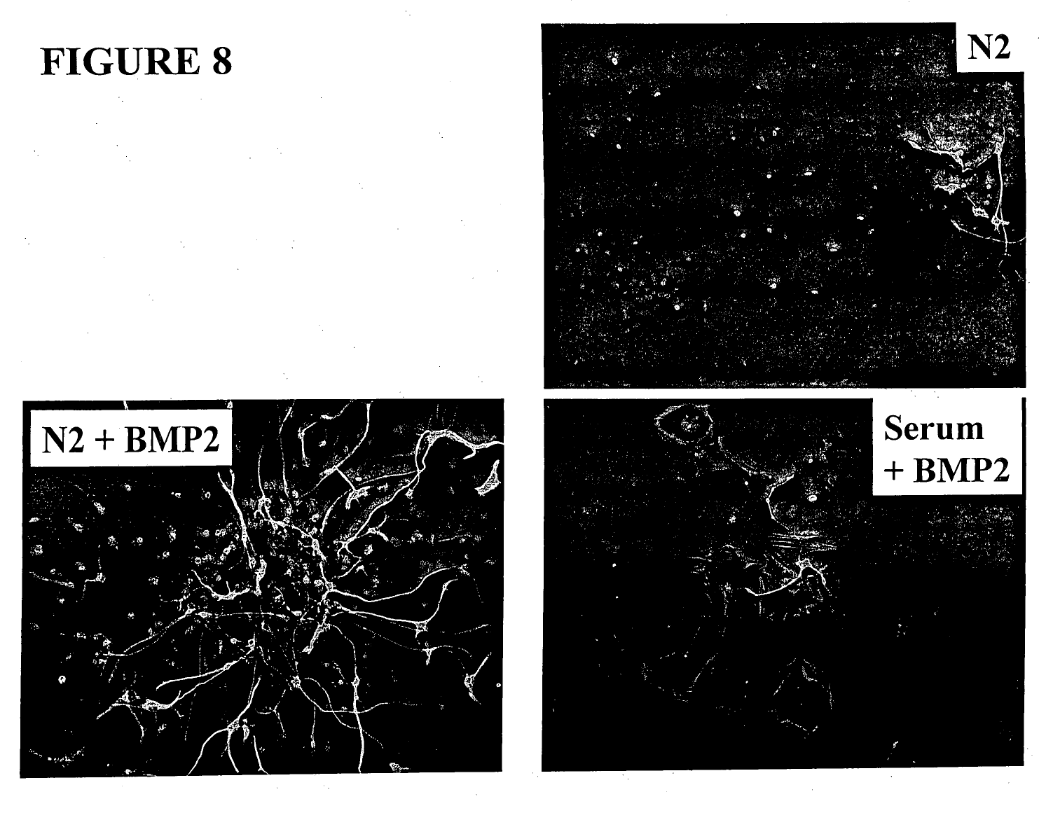

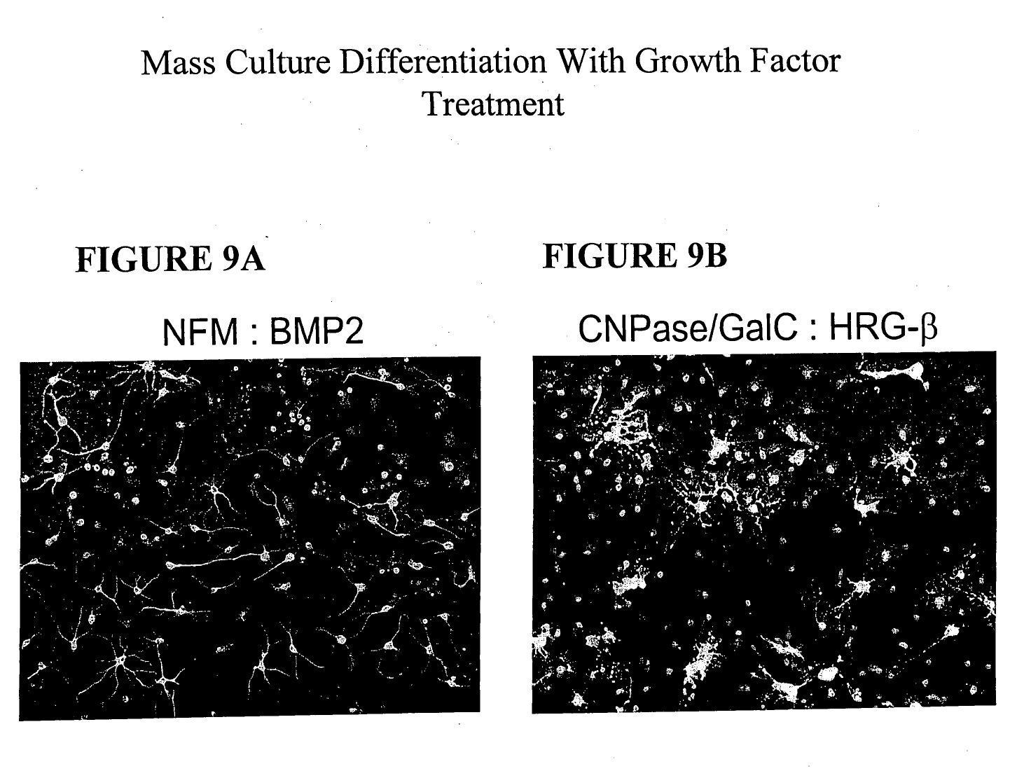

NC-derived spheres were plated on poly-D-lysine and laminin in DMEM/F12 supplemented with chick extract, N2, retinoic acid, NGF and 2% serum. Immunostaining was performed for neurofilament after 6 days (see FIG. 7). NC-derived spheres were also plated on poly-D-lysine and laminin in DMEM/F12 supplemented with N2, N2+BMP2, or Serum+BMP2. Differentiation was assessed by neurofilament and smooth muscle actin (SMA) staining (see FIG. 8). Our results show that like NCSCs, cells from the NC-derived spheres differentiated into neurons and smooth muscle cells following BMP2 and serum treatment, respectively.

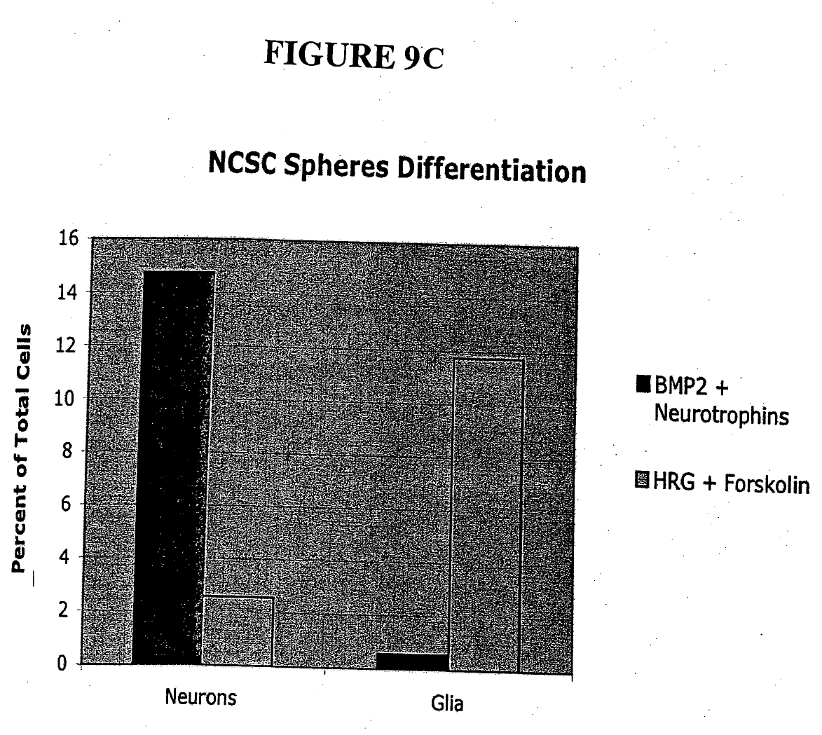

NC-derived spheres were passaged five times in DMEM:F12 supplemented with chick extract, N2, retinoic acid, NGF, and 2% serum. NC-derived spheres were treated with either BMP2 or HRG-β, after which immunostaining was performed. Differentiation was assessed by neurofilament and CNPase/Gal C staining (see FIGS. 9A and 9B). As shown above, BMP2 induced neuronal differentiation, whereas HRG-β induced glial differentiation. FIG. 9C is a table representing the number of neuronal and glial cells that are produced by culturing NCSCs in the presence of BMP2 or HRG-β.

### OTHER EMBODIMENTS

All publications and patent applications cited in this specification are herein incorporated by reference as if each individual publication or patent application were specifically and individually indicated to be incorporated by reference. Although the foregoing invention has been described in some detail by way of illustration and example for purposes of clarity of understanding, it will be readily apparent to those of ordinary skill in the art in light of the teachings of this invention that certain changes and modifications may be made thereto without departing from the spirit or scope of the appended claims.

What is claimed is:

1. A mammalian neural crest stem cell (NCSC) capable of producing non-neuronal and neuronal cells, wherein said NCSC expresses PSA-NCAM, and nestin, and optionally expresses p75.

2. The NCSC of claim 1, wherein NCSC expresses one or more proteins selected from the group consisting of FGFR, CD44, S100β, Pax3, twist, and fibronectin.

3. The NCSC of claim 1, wherein said NCSC is capable of producing a neuron, Schwann cell, oligodendrocyte, astrocyte, cardiomyocyte, lung cell, adipocyte, pancreatic islet cell, hematopoeitic cell, kidney cell, hepatocyte, chondrocyte, epithelial cell, endothelial cell, skeletal muscle cell, melanocyte, smooth muscle cell, cartilage, or connective tissue under appropriate conditions.

4. The NCSC of claim 1, wherein said NCSC is obtained by a method comprising the steps of:
(a) culturing a mammalian tissue containing NCSCs in a first culture for a period of at least two days under conditions in which NCSCs adhere to the culture surface;
(b) transferring adherent cells from step (a) to a second culture under conditions in which NCSCs grow non-adherently and non-NCSCs grow adherently or die; and
(c) collecting nonadherent cells.

5. The NCSC of claim 1, wherein said NCSC is obtained by a method that does not employ an antibody specific to p75.

6. The NCSC of claim 1, wherein said NCSC is isolated from embryonic tissue.

7. The NCSC of claim 6, wherein said embryonic tissue is the neural tube.

8. The NCSC of claim 1, wherein said NCSC is isolated from tissue from a post-natal mammal.

9. The NCSC of claim 8, wherein said tissue is the gastro-intestinal tract.

10. The NCSC of claim 1, wherein said NCSC is isolated from a human.

11. The NCSC of claim 1, wherein said NCSC expresses a heterologous gene.

12. The NCSC of claim 11 wherein said heterologous gene is in an expression vector.

13. A population of at least 10 cells, wherein at least 30% of said cells are mammalian NCSCs capable of producing non-neuronal and neuronal cells, wherein at least 50% of said NCSCs express PSA-NCAM, and nestin, and optionally express p75.

14. The population of claim 13, wherein said NCSCs express one or more proteins selected from the group consisting of FGFR, CD44, S100β, Pax3, twist, and fibronectin.

15. The population of claim 13, wherein said NCSCs are capable of producing neurons, Schwann cells, oligodendrocytes, astrocytes, cardiomyocytes, lung cells, adipocytes, pancreatic islet cells, hematopoeitic cells, kidney cells, hepatocytes, chondrocytes, epithelial cells, endothelial cells, skeletal muscle cells, melanocytes, smooth muscle cells, cartilage, or connective tissue under appropriate conditions.

16. A method for obtaining a self-renewing mammalian NCSC capable of producing non-neuronal and neuronal cells, said method comprising the steps of:
(a) culturing a mammalian tissue containing NCSCs in a first culture for a period of at least two days under conditions in which NCSCs adhere to the culture surface;
(b) transferring adherent cells from step (a) to a second culture under conditions in which NCSCs grow non-adherently and non-NCSCs grow adherently or die; and
(c) collecting nonadherent cells.
